SEQUENCE LISTING (7 sequences)

SEQ ID NO: 1 - protein, 76 aa, Homo sapiens
QPDAINAPVTCCYNFTNRKISVQRLASYRRITSSKCPKEAVIFKTIVAKEICADPKQKWVQDSMDHLDKQTQTPKT

SEQ ID NO: 2 - protein, 76 aa, Artificial Sequence
  MCP1 mutant M1
QPDAINAPVTCCYNFTKRKIKVQRLASYRRITSSKCPKEAVIFKTIVAKEICADPKQKWVQDSMDHLDKQTQTPKT

SEQ ID NO: 3 - protein, 76 aa, Artificial Sequence
  MCP1 mutant M2
QPDAINAPVTCCYNFTKRKISVKRLASYRRITSSKCPKEAVIFKTIVAKEICADPKQKWVQDSMDHLDKQTQTPKT

SEQ ID NO: 4 - protein, 76 aa, Artificial Sequence
  MCP1 mutant M3
QPDAINAPVTCCYNFTKRKISVQRLASYRRITSSKCPKEAVIFKTIKAKEICADPKQKWVQDSMDHLDKQTQTPKT

SEQ ID NO: 5 - protein, 76 aa, Artificial Sequence
  MCP1 mutant M4
QPDAINAPVTCCYNFTKRKISVQRLASYRRITSKKCPKEAVIFKTIKAKEICADPKQKWVQDSMDHLDKQTQTPKT

SEQ ID NO: 6 - protein, 76 aa, Artificial Sequence
  MCP1 mutant M5
QPDAINAPVTCCYNFTKRKIKVQRLASYRRITSKKCPKEAVIFKTIKAKEICADPKQKWVQDSMDHLDKQTQTPKT

SEQ ID NO: 7 - protein, 77 aa, Artificial Sequence
  MCP1 mutant protein formula
XQPDAINAXVTCCXNFTXXXIXVXRLASYRRITSXKCPKEAVIFKTIXAKEICADPKQKWVQDSMDHLDKQTQTPKT

(19) United States
(12) Patent Application Publication
(10) Pub. No.: US 2012/0046218 A1
(43) Pub. Date: Feb. 23, 2012

(54) GLYCOSAMINOGLYCAN-ANTAGONISING MCP-I MUTANTS AND METHODS OF USING SAME

(75) Inventor: Andreas Kungl, Graz (AT)

(73) Assignee: PROTAFFIN BIOTECHNOLOGIE AG, Graz (AT)

(21) Appl. No.: 13/145,217

(22) PCT Filed: Jan. 29, 2010
(86) PCT No.: PCT/EP10/51128

§ 371 Date: Jul. 19, 2011

Related U.S. Application Data
(60) Provisional application No. 61/148,766, filed on Jan. 30, 2009.

(30) Foreign Application Priority Data
Jul. 27, 2009 (EP) 09166429.2

(51) Int. Cl. A61K 38/19; C12N 15/19; A61P 29/00; A61P 27/02; A61P 9/10; C12N 1/21; A61P 37/00; A61P 1/00; A61P 9/04; A61K 31/711; C12N 15/70; C07K 14/52; A61P 25/00

(52) U.S. Cl. 514/1.9; 530/324; 536/23.5; 514/12.2; 514/16.6; 514/20.8; 514/15.1; 514/17.9; 514/44 R; 435/320.1; 435/252.33

(57) ABSTRACT

Novel mutants of human monocyte chemoattractant protein 1 (MCP-1) with increased glycosaminoglycan (GAG) binding affinity and knocked-out or reduced GPCR activity compared to wild type MCP-1, and their use for therapeutic treatment of inflammatory diseases.

The present invention relates to novel mutants of human monocyte chemoattractant protein 1 (MCP-1) with increased glycosaminoglycan (GAG) binding affinity and knocked-out or reduced GPCR activity compared to wild type MCP-1, and to their use for therapeutic treatment of inflammatory diseases.

All chemokines, with the exception of lymphotactin and fraktaline/neurotactin which are members of the C and CX3C chemokine subfamily, respectively, have four cysteines in conserved positions and can be divided into the CXC or α-chemokine and the CC or β-chemokine subfamilies on the basis of the presence or absence, respectively, of an amino acid between the two cysteines within the N-terminus. Chemokines are small secreted proteins that function as intercellular messengers to orchestrate activation and migration of specific types of leukocytes from the lumen of blood vessels into tissues (Baggiolini M., J. Int. Med. 250, 91-104 (2001)). This event is mediated by the interaction of chemokines with seven transmembrane G-protein-coupled receptors (GPCRs) on the surface of target cells. Such interaction occurs in vivo under flow conditions. Therefore, the establishment of a local concentration gradient is required and ensured by the interaction of chemokines with cell surface glycosaminoglycans (GAGs). Chemokines have two major sites of interaction with their receptors, one in the N-terminal domain which functions as a triggering domain, and the other within the exposed loop after the second cysteine, which functions as a docking domain (Gupta S. K. et al., Proc. Natl. Acad. Sci., USA, 92, (17), 7799-7803 (1995)). The GAG binding sites of chemokines comprise clusters of basic amino acids spatially distinct (Ali S. et al., Biochem. J. 358, 737-745 (2001)). Some chemokines, such as RANTES, have the BBXB motif in the 40s loop as major GAG binding site; IL-8 interacts with GAGs through the C-terminal α-helix and Lys 20 in the proximal N-loop. Other chemokines, such as MCP-1, show a significant overlap between the residues that comprise the receptor binding and the GAG binding site (Lau E. K. et al., J. Biol. Chem., 279 (21), 22294-22305 (2004)).

In the context of the chemokine-β family of cytokines, monocyte chemoattractant protein-1 (MCP-1) is a monocyte and lymphocyte-specific chemoattractant and activator found in a variety of diseases that feature a monocyte-rich inflammatory component, such as atherosclerosis (Nelken N. A. et al., J. Clin. Invest. 88, 1121-1127 (1991); Yla-Herttuala, S., Proc. Natl. Acad. Sci. USA 88, 5252-5256 (1991), rheumatoid arthritis (Koch A. E. et al., J. Clin. Invest. 90, 772-779 (1992); Hosaka S. et al., Clin. Exp. Immunol. 97(3), 451-457 (1994), Robinson E. et al., Clin. Exp. Immunol. 101(3), 398-407 (1995)), inflammatory bowel disease (MacDermott R. P. et al., J. Clin. Immunol. 19, 266-272 (1999)) and congestive heart failure (Aukrust P., et al., Circulation 97, 1136-1143 (1998), Hohensinner P. J. et al., FEBS Letters 580, 3532-3538 (2006)). Crucially, knockout mice that lack MCP-1 or its receptor CCR2, are unable to recruit monocytes and T-cells to inflammatory lesions (Grewal I. S. et al., J. Immunol. 159 (1), 401-408 (1997), Boring L. et al., J. Biol. Chem. 271 (13), 7551-7558 (1996), Kuziel W. A., et al., Proc. Natl. Acad. Sci. USA 94 (22), 12053-8 (1997), Lu B., et al., J. Exp. Med. 187 (4), 601-8 (1998)); furthermore, treatment with MCP-1 neutralizing antibodies or other biological antagonists can reduce inflammation in several animal models (Lukacs N. W. et al., J. Immunol., 158 (9), 4398-4404 (1997), Flory C. M. et al., 1. Lab. Invest. 69 (4), 396-404 (1993), Gong J. H., et al., J. Exp. Med. 186 (1), 131-7 (1997), Zisman D. A. et al., J. Clin. Invest. 99 (12), 2832-6 (1997)). Finally, LDL-receptor/MCP-1-deficient and apoB-trans-genic/MCP-1-deficient mice show considerably less lipid deposition and macrophage accumulation throughout their aortas compared to the WT MCP-1 strains (Alcami A. et al., J. Immunol. 160 (2), 624-33 (1998), Gosling J. et al., J. Clin. Invest. 103 (6), 773-8 (1999)).

Potzinger et al. (Biochemical Society Transactions, 34, 2006, 435-437) describes the generation of modified IL-8 proteins as protein-based GAG antagonists in an inflammatory setting.

Piccinini et al. have shown the effect of a limited number of site-directed MCP-1 mutants on enhanced glycosaminoglycan binding (J Biol. Chem. 2010 Jan. 22. [Epub ahead of print]).

Proudfoot et al. (Proc. Natl. Acad. Sci., 100, 4, 2003, 1885-1890) investigated the effect of mutations in the GAG binding sites of chemokines, amongst others of MCP-1. The specific mutant (18AA19)-MCP-1 shows only residual affinity for heparin.

US2003/0162737 discloses an antagonistic MCP-1 mutein for the treatment of pulmonary hypertension. Said MCP-1 mutein comprises several deletions at the N-terminus of the protein, up to deletion of N-terminal amino acids 1-10 or 2-8. Further the mutein can comprise a modification at amino acid positions 22 or 24.

Steitz S. et al. (FEBS Letters, 430, 3, 1998, 158-164) investigated the role of N-terminal modifications on receptor binding. MCP-1 mutants comprising substitutions of amino acid positions 13 and 18 were disclosed. Y13A showed a dramatic loss in function to induce THP-1 chemotaxis.

Lubkowski J. et al. (Nature Structural Biology, 4, 1, 1997, 64.69) investigated the x-ray crystal structure of recombinant human MCP-1. The N-terminus of the protein was modified and its effect on activity was measured. It was shown that modification specifically at positions 10 and 13 lowered the activity of MCP-1 and had an effect on the dimer stabilization. An impaired chemotactic activity of the mutants suggested a functional significance for Tyr28, Arg 29, Arg30 and Asp68, It was noted that charged amino acids (Arg, Asp) destabilize an alternate dimer and that the introduction of uncharged residues can significantly increase stability.

Since the first chemokines and their receptors have been identified, the interest on exactly understanding their roles in normal and diseased physiology has become more and more intense. The constant need for new anti-inflammatory drugs with modes of action different from those of existing drugs support the development of new protein-based GAG-antagonists and their use in an inflammatory set.

In the last years the molecular basis of the interactions of MCP-1 with CCR2 and GAGs have been studied in great detail, targeted engineering of the chemokine towards becoming an effective antagonist of MCP-1's biological action is feasible.

For this purpose several recombinant MCP-1 variants that compete with their wild type counterpart for glycosaminoglycan binding and show reduced or knocked out activation of leukocytes have been generated.

Consequently, one subject matter of the present invention is to inhibit leukocyte, more specifically monocyte and T cell, migration by antagonizing the GAG interaction with an MCP-1-based mutant protein specifically in the context of inflammatory or allergic processes.

MCP-1 mutants with a higher GAG binding affinity either by modifying the wild type GAG binding region or by introducing a new GAG binding region into the MCP1 protein and simultaneously knocking out or reducing its GPCR activity, specifically the CCR2 activity of the chemokine have been described in WO2009/015884A1. WO2009/015884A1 describes MCP-1 proteins wherein a region of the MCP-1 protein is modified in a structure conserving way by introducing basic and/or electron donating amino acids or replacing native amino acids with basic and/or electron donating amino acids and optionally also modifying the N-terminal region of said MCP-1 protein by addition, deletion and/or replacement of amino acids and, optionally, adding an N-terminal Methionine (M) to the mutant MCP-1 protein, resulting in partial or complete loss of chemotactic activity have been disclosed there. Said MCP-1 mutants can specifically exhibit a minimum five-fold improved Kd for standard GAGs (heparin or heparan sulfate) and they are deficient or reduced in inducing Calcium-release in standard monocytic cell culture.

According to the present invention novel MCP-1 mutants having a GAG binding affinity that is even more increased were developed exhibiting a minimum six-fold improved Kd for standard GAGs (heparin or heparan sulfate) compared to wild type This is achieved by specifically modifying amino acids at positions 17 and/or 34 of the wild type protein. According to a specific embodiment of the invention, the MCP-1 mutant also comprises a modification of amino acid position 21.

The mutant MCP-1 proteins according to the present invention can also be formulated as a pharmaceutical composition comprising the mutant MCP-1 protein or a polynucleic acid molecule coding for MCP-1 mutant protein, a vector containing an isolated DNA molecule coding for the MCP-1 mutant protein, and a pharmaceutically acceptable carrier.

Said MCP-1 mutant protein or the polynucleotide coding therefore or the vector containing said polynucleotide can also be used for inhibiting or suppressing the biological activity of the respective wild type protein.

The inventive MCP-1 mutant protein according to the invention can also be used in a method for preparing a medicament for the treatment of chronic or acute inflammatory diseases or allergic conditions. Preferably, the disease is selected from the group comprising rheumatoid arthritis, uveitis, inflammatory bowel disease, myocardial infarction, congested heart failure, diabetic complications (such as retinopathy, necropathy, etc.), multiple sclerosis or ischemia reperfusion injury.

### FIGURES

FIG. 1: Sequence of MCP-1 mutants, mutations with respect to the wild type chemokine are underlined

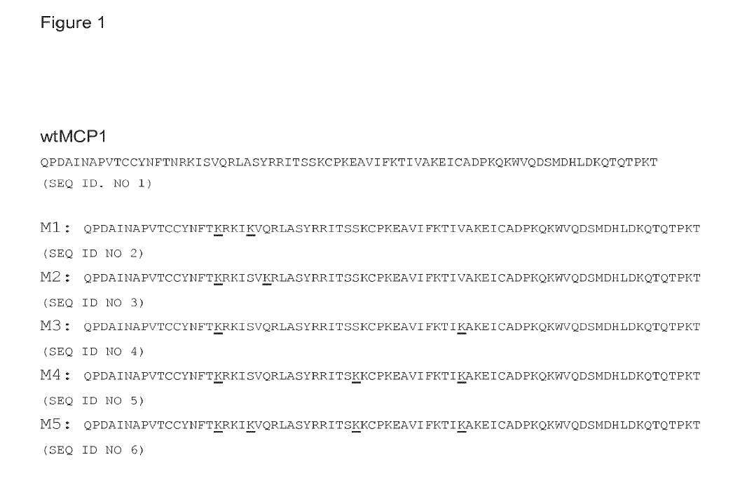

FIG. 2: Affinities (expressed as dissociation constants Kd) of MCP-1 and MCP-1 mutants with respect to the natural glycosaminoglycan ligand heparan sulfate.

FIG. 3: Surface Plasmon Resonance Analysis-Binding of wtMCP-1/CCL2, Met-MCP-1(Y13AS21K) and Met-MCP-1(Y13A521KQ23R) to unfractionated heparan sulfate

It has been shown that increased GAG binding affinity can be introduced by increasing the relative amount of basic and/or electron donating amino acids in the GAG binding region (also described in WO 05/054285, incorporated in total herein by reference), leading to a modified protein that acts as competitor with natural GAG binding proteins. This was particularly shown for interleukin-8. The specific location of GAG binding regions and their modification by selectively introducing at least two basic and/or electron donating amino acids was not disclosed for MCP-1 protein.

Additionally, the amino terminus of MCP-1 was found to be essential for chemokine signalling through its GPC receptor CCR2. In order to engineer an MCP-1-based CCR2 antagonist, others have engineered MCP-1 in a way to completely knock-out GAG binding and to leave CCR2 binding intact (WO03084993A1). By these means, it was intended to block MCP-1-mediated signalling by blocking the CCR2 receptor on neutrophils and to prevent attachment on the endothelium via the GAG chains. It was therefore not obvious to turn this approach around by blocking the GAG chains on the endothelium (by engineering higher GAG binding affinity) and to knock out the CCR2 binding of MCP-1.

The invention now provides a novel MCP1 mutant protein with increased GAG binding affinity and reduced GPCR activity compared to wild type MCP-1 protein, characterized in that the MCP-1 protein is modified in a structure-conserving way by replacement of at least two amino acids by basic and/or electron donating amino acids wherein at least one amino acid at positions 17 or 34 is replaced and optionally at least one amino acid at positions 21, 23 or 47 is replaced. Preferably, the mutant comprises modifications at both positions 17 and 34. More preferred, the mutant comprises a further modification at position 21.

Alternatively, the mutant comprises a modification at one of positions 17 or 34 in combination with a modification at position 21.

By substituting amino acids at positions 17 and/or 34 the GAG binding affinity can be potentially further increased by a factor of >1.5; preferably >2 compared to known MCP1 mutant proteins. These positions were surprisingly found to be highly relevant in view of GAG binding affinity since two sulfate ions were discovered in the vicinity of these residues in one of the published MCP-1 crystal structures. A potential second binding site was successfully modelled within MCP-1 for GAGs. Therefore, introducing further basic amino acids into these sites by the proposed method increases not only the potential for higher GAG binding affinity but might also extend the mode of action of these mutants in therapeutic settings.

According to a specific embodiment, the modified MCP-1 protein further comprises a further modification of at least one amino acid of the first 1 to 10 amino acids of the N-terminal region of said MCP-1 protein by addition, deletion and/or replacement of at least one amino acid residue.

If the native amino acids replaced by said basic or electron donating amino acids are basic amino acids, the substituting amino acids have to be more basic amino acids or comprise a different structural flexibility compared to the native amino acid residue. Structural flexibility according to the invention is defined by the degree of accommodating to an induced fit as a consequence of GAG ligand binding.

According to a specific embodiment of the invention the native amino acids replaced by basic and/or electron donating amino acids are non-basic amino acids.

According to the definition as used in the present application the term MCP-1 mutant protein can also include any parts or fragments thereof that still show chemokine-like fold but impacts on/knocks out chemokine activity like monocyte or T-cell chemotaxis and Ca-release.

The term “vicinity” as defined according to the invention comprises amino acid residues which are located within the conformational neighbourhood of the GAG binding site but not positioned at the GAG binding sites. Conformational neighbourhood can be defined as either amino acid residues which are located adjacent to GAG binding amino acid residues in the amino acid sequence of a protein or amino acids which are conformationally adjacent due to three dimensional structure or folding of the protein.

The term “adjacent” according to the invention is defined as lying within the cut-off radius of the respective amino acid residues to be modified of not more than 20 nm, preferably 15 nm, preferably 10 nm, preferably 5 nm.

To be able to perform their biological function, proteins fold into one, or more, specific spatial conformations, driven by a number of noncovalent interactions such as hydrogen bonding, ionic interactions, Van der Waals' forces and hydrophobic packing. Three dimensional structure can be determined by known methods like X-ray crystallography or NMR spectroscopy.

Identification of native GAG binding sites can be determined by mutagenesis experiments. GAG binding sites of proteins are characterized by basic residues located at the surface of the proteins. To test whether these regions define a GAG binding site, these basic amino acid residues can be mutagenized and decrease of heparin binding affinity can be measured. This can be performed by any affinity measurement techniques as known in the art.

Rational designed mutagenesis by insertion or substitution of basic or electron-donating amino acids can be performed to introduce foreign amino acids in the vicinity of the native GAG binding sites which can result in an increased size of the GAG binding site and in an increase of GAG binding affinity. The size can be increased by at least one additional amino acid introduced into the MCP-1 protein, specifically by introduction of at least two amino acids, more specifically of at least three amino acids.

A deviation of the modified structure as measured by far-UV CD spectroscopy from wild type MCP-1 structure of less than 30%, preferably less than 20%, preferably less than 10% is defined as structure conserving modification according to the invention.

According to an alternative embodiment, the structure conserving modification is not located within the N-terminus of the MCP1 protein.

The key residues relating to the GAG binding domain of wtMCP-1 are N17, S21, Q23, S34 and/or V47. At least one amino acid at positions 17 or 34 and optionally at least one amino acid at positions 21, 23 or 47 have to be modified by insertion of a basic and/or electron donating amino acid. If positions 17 and 34, optionally in combination with at least one additional modification, are modified, GAG binding affinity is even more increased compared to single modifications at these positions.

The inventive MCP-1 protein can comprise any combinations of amino acid modifications at positions N17, S21, Q23, S34 and/or V47 resulting in an MCP-1 mutant protein having increased GAG binding compared to wt MCP-1.

Specifically, all amino acids at positions 17, 21, 23, 34 and 47 can be modified according to the invention.

The modifications can be, for example, a substitution of, or replacement by, at least two basic or electron donating amino acids. Electron donating amino acids are those amino acids which donate electrons or hydrogen atoms (Droenstedt definition). Specifically, these amino acids can be N or Q. Basic amino acids can be selected from the group consisting of R, K and H.

According to a further embodiment of the invention, R at amino acid position 18 can by modified by K, and/or K at amino acid position 19 can be modified by R and/or P8 can be modified by any amino acid substitution to receive at least partially decrease receptor binding of the modified MCP-1.

Alternatively, the MCP-1 mutant protein of the invention is characterized in that Y at position 13 is further substituted by any amino acid residue, preferably by A.

Y13 and R18 were shown to be also critical residues for signalling, and the replacement of these residues by other amino acid residues gave rise to a protein unable to induce chemotaxis. Two-dimensional 1H-15N HSQC spectra recorded on both deletion and substitution MCP-1 variants revealed that these mutations do not generate misfolded proteins (Chad D. Paavola et al., J. Biol. Chem., 273 (50), 33157-33165 (1998)).

Furthermore, the N-terminal methionine reduces the binding affinity of MCP-1 for CCR2 on THP-1 cells (Hemmerich S. et al, Biochemistry 38 (40), 13013-13025 (1999)) so that the chemotactic potency of [Met]-MCP-1 is approximately 300-fold lower than of the wild type (Jarnagin K. et al., Biochemistry 38, 16167-16177 (1999)). This is in contrast to the potent receptor antagonist [Met]-RANTES which does not induce chemotaxis but binds with high affinity to the receptor.

Therefore, according to an alternative embodiment of the invention, the MCP-1 mutant protein may contain an N-terminal Met. MCP-1 variants retaining the N-terminal methionine appear to have an increased apparent affinity for heparin (Lau E. K. et al., J. Biol. Chem. 279 (21), 22294-22305 (2004)).

According to the present invention, the N-terminal region of the wild type MCP-1 region that can be modified comprises the first 1 to 10 N-terminal amino acids. The inventive MCP-1 mutant protein can also have the N-terminal amino acid residues 2-8 deleted. Truncation of residues 2-8 ([1+9-76]hMCP-1) produces a protein that cannot induce chemotaxis.

In order to knock out GPCR activity and at the same time to improve affinity for GAGs, minimizing the number of modifications as far as possible, site-directed MCP-1 mutants were designed using bioinformatical and biostructural tools. This means, since the structure of wtMCP-1 is known, mutants were rationally designed. This means for knocking-in higher GAG binding affinity, that more GAG binding sites are introduced into the already existing GAG binding domain by replacing amino acids which are not directly involved in GAG binding, which are structurally less important, and which are solvent exposed by vicinity to basic amino acids such as K or R. By doing so, special attention was drawn to conserving the specific GAG interaction sites of MCP-1, i.e. those amino acids responsible for hydrogen bonding and van der Waals contacts with the GAG ligand, as well as the overall fold of the chemokine to preserve the ability of the chemokine to penetrate chemokine networks which relies on protein-protein interactions contained in the surface of MCP-1.

The amino acid sequence of the modified MCP-1 molecule can be described by the general formula:

MCP-1 mutant protein, characterized in that it comprises the amino acid sequence of the general formula:

(M)ₙQ(PDAINA(Z1))ₘVTCC(X1)NFT(X2)(Z2)(Z3)I(X3)V(X4)RLASYRRITS(X5)
KCPKEAVIFKTI(X6) AKEICADPKQ KWVQDSMDHL DKQTQTPKT

wherein Z1 is selected from the group of P and A, G, L, preferably it is A,
wherein Z2 is selected from the group of R and K,
wherein Z3 is selected from the group of K and R,
wherein X1 is selected from the group consisting of Y and/or A, preferably it is A,
wherein X2 is selected from the group consisting of N, R, K, H, N or Q, preferably it is K,
wherein X3 is selected from the group consisting of S, K, H, N and/or Q, preferably it is K,
wherein X4 is selected from the group consisting of R, K, H, N and/or Q, preferably it is K or R,
wherein X5 is selected from the group consisting of S, K, H, N and/or Q, preferably it is K,
wherein X6 is selected from the group consisting of V, R, K, H, N and/or Q, preferably it is K,
and wherein n and/or m can be either 0 or 1
and wherein at least two of amino acids X1 to X6 are modified with the proviso that at least one of positions X2 or X5 and optionally at least one of positions X1, X3 or X4 are modified.

Specifically the inventive MCP-1 mutant protein can be selected from the group of Met-MCP-1 Y13A N17K S21K Q23K V47K, Met-MCP-1 Y13A N17K S21K S34K, Met-MCP-1 Y13A N17K S21K Q23K S34K and Met-MCP-1 Y13A S21K Q23K S34K V47K.

A further aspect of the present invention is an isolated polynucleic acid molecule which codes for the inventive protein as described above.

The polynucleic acid may be DNA or RNA. Thereby the modifications which lead to the inventive MCP-1 mutant protein are carried out on DNA or RNA level. This inventive isolated polynucleic acid molecule is suitable for diagnostic methods as well as gene therapy and the production of inventive MCP-1 mutant protein on a large scale.

A further aspect relates to a vector comprising an isolated DNA molecule according to the present invention, as defined above. The vector comprises all regulatory elements necessary for efficient transfection as well as efficient expression of proteins. Such vectors are well known in the art and any suitable vector can be selected for this purpose.

A further aspect of the present invention relates to a recombinant cell, specifically a non-human cell which is transfected with an inventive vector as described above. Transfection of cells and cultivation of recombinant cells can be performed as well known in the art. Such a recombinant cell as well as any descendant cell therefrom comprises the vector. Thereby, a cell line is provided which expresses the MCP-1 mutant protein either continuously or upon activation depending on the vector.

A further aspect of the invention relates to a pharmaceutical composition comprising a MCP-1 mutant protein, a polynucleic acid or a vector according to the present invention, as defined above, and a pharmaceutically acceptable carrier. Of course, the pharmaceutical composition may further comprise additional substances which are usually present in pharmaceutical compositions, such as salts, buffers, emulgators, coloring agents, etc.

The pharmaceutical composition can be administered by any route as known in the art, specifically by oral, subcutaneous, intravenous, intramuscular administration or by inhalation.

A further aspect of the present invention relates to the use of the MCP-1 protein, a polynucleic acid or a vector according to the present invention, as defined above, in a method for either in vivo or in vitro inhibiting or suppressing the biological activity of the respective wild type protein. As mentioned above, the MCP-1 mutant protein of the invention will act as an antagonist whereby the side effects which occur with known recombinant proteins will not occur with the inventive MCP-1 mutant protein. In this case this will particularly be the biological activity involved in inflammatory reactions.

Therefore, a further use of the MCP-1 protein, a polynucleic acid or a vector according to the present invention, as defined above, is in a method for producing a medicament for the treatment of an inflammatory condition. In particular, it will act as antagonist without or with reduced side effects and will be particularly suitable for the treatment of inflammatory diseases or conditions, either of chronic or acute nature. Therefore, a further aspect of the present invention is also a method for the treatment of inflammatory diseases or allergic conditions, wherein the MCP-1 mutant protein according to the invention, the isolated polynucleic acid molecule or vector according to the present invention or a pharmaceutical preparation according to the invention is administered to a patient.

More specifically, the inflammatory diseases or allergic conditions are respiratory allergic diseases such as asthma, allergic rhinitis, COPD, hypersensitivity lung diseases, hypersensitivity pneumonitis, interstitial lung disease, (e.g. idiopathic pulmonary fibrosis, or associated with autoimmune diseases), anaphylaxis or hypersensitivity responses, drug allergies and insect sting allergies; inflammatory bowel diseases, such as Crohn's disease and ulcerative colitis; spondyloarthropathies, scleroderma; psoriasis and inflammatory dermatoses such as dermatitis, eczema, atopic dermatitis, allergic contact dermatitis, uticaria; vasculitis; autoimmune diseases with an aetiology including an inflammatory component such as arthritis (for example rheumatoid arthritis, arthritis chronica progrediente, psoriatic arthritis and arthritis deformans) and rheumatic diseases, including inflammatory conditions and rheumatic diseases involving bone loss, inflammatory pain, hypersensitivity (including both airways hypersensitivity and dermal hypersensitivity) and allergies. Specific autoimmune diseases include autoimmune hematological disorders (including e.g. hemolytic anaemia, aplastic anaemia, pure red cell anaemia and idiopathic thrombocytopenia), systemic lupus erythromatosus, polychondritis, Wegener granulomatosis, dermatomyositis, chronic active hepatitis, myasthenia gravis, psoriasis, Steven-Johnson syndrome, autoimmune inflammatory bowel disease (including e.g. ulcerative colitis, Crohn's disease and Irritable Bowel Syndrome), autoimmune thyroiditis, Behcet's disease, endocrine ophthalmopathy, Graves disease, sarcoidosis, multiple sclerosis, primary biliary cirrhosis, juvenile diabetes (diabetes mellitus type I), uveitis (anterior and posterior), keratoconjunctivitis sicca and vernal keratoconjunctivitis, interstitial lung fibrosis, and glomerulonephritis (with and without nephrotic syndrome, e.g. including idiopathic nephrotic syndrome or minimal change nephropathy); graft rejection (e.g. in transplantation including heart, lung, combined heart-lung, liver, kidney, pancreatic, skin, or corneal transplants) including allograft rejection or xenograft rejection or graft-versus-host disease, and organ transplant associated arteriosclerosis; atherosclerosis; cancer with leukocyte infiltration of the skin or organs; stenosis or restenosis of the vasculature, particularly of the arteries, e.g. the coronary artery, including stenosis or restenosis which results from vascular intervention, as well as neointimal hyperplasia; and other diseases or conditions involving inflammatory responses including ischemia reperfusion injury, hematologic malignancies, cytokine induced toxicity (e.g. septic shock or endotoxic shock), diabetic complications such as retinopathy, necropathy, etc., polymyositis, dermatomyositis, and granulomatous diseases including sarcoidosis.

Preferably, the inflammatory disease is selected form the group comprising rheumatoid arthritis, uveitis, inflammatory bowel disease, myocardial infarction, congested heart failure or ischemia reperfusion injury, diabetic complications like retinopathy or neuropathy, multiple sclerosis and artherosclerosis.

The following examples describe the invention in more detail without limiting the scope of the invention.

### EXAMPLES

The mutants are subjected to
Isothermal fluorescence titrations (IFTs) to study the enhanced binding affinity to glycosaminoglycan ligands such as heparan sulfate. Since this method is based on non-immobilised ligands, the results are complementary to the SPR method outlined below.
Surface plasmon resonance (SPR) experiments to investigate their improved binding affinity as well as their improved binding kinetics with respect to the wild type protein. For this purpose, the potential GAG ligand (heparan sulfate or chondroitin sulfate) is immobilised on an SPR chip and the interaction is quantitated in a constant flow experiment.
In competition assays using fluorescently labelled wtMCP-1 as well as further chemokines the competitive advantage of the new MCP-1 mutants over already existing MCP-1 mutants and wild type are investigated.

### Example 1

The gene for WT MCP-1 was constructed by standard gene synthesis techniques with optimal codon usage for expression in E. coli and was cloned into a pJExpress 411 vector (1). Features of the vector include a T7 promoter for high-level, IPTG-inducible expression of the gene in E. coli and a kanamycin resistance marker for selection in E. coli. Each mutation was introduced by de novo synthesising the corresponding gene and incorporation again in the pJExpress 411 expression vector. The constructs were checked by DNA sequencing before protein expression. The pJExpress 411 plasmids containing the MCP-1 mutant genes were transformed into E. coli strain BL21-DE3. Starting cultures were prepared and used for protein expression. Cultures were grown in 3 L Erlenmeyer flasks under shaking at 37° C. in LB broth containing 30 μg/ml kanamycin to an OD₆₀₀ of 0.8. Protein expression was induced by addition of 1.0 mM isopropyl-β-D-thiogalactopyranoside. Cells were incubated with shaking for an additional 3-4 hours and harvested by centrifugation for 10-15 minutes at 6,000×g. Each g wet cell pellet was resuspended in 4 ml solution buffer, containing 10 mM KH₂PO₄ pH 7.5, and disrupted by sonication on ice. The lysate was then cleared by centrifugation for 20 minutes at 20,000×g at 4° C. The inclusion body pellets were solubilised in 10 ml solution buffer, containing 10 mM KH₂PO₄ pH 7.5, guanidinium hydrochloride 6M, per g wet cell pellet under stirring for 3 hours at room temperature. After centrifugation for 20 minutes at 20,000×g at 4° C. the supernatant was dialyzed against 10 mM KH₂PO₄ pH 7.5 at 4° C. The precipitant was spun down and the solution was loaded on a SP-Sepharose high performance column (Amersham Bioscience). Mutants were eluted with a linear gradient from 10 mM KH₂PO₄ pH 7.5 to 10 mM KH₂PO₄ pH 7.5, 1M NaCl over 75 minutes with a flow rate of 2 ml/min; the MCP-1 mutants eluted at 0.6-0.8 M NaCl. Peak fractions were pooled and purified by reversed-phase HPLC on a C18 column. The mutants were eluted with a non linear gradient: from 10% to 40% acetonitrile (0.1% TFA) in 5 minutes, from 40% to 60% acetonitrile (0.1% TFA) in 20 minutes and from 60% to 90% acetonitrile (0.1% TFA) in 5 minutes with a flow rate of 10 ml/min. Proteins eluted at 48±5% acetonitrile and were refolded by loading them on a SP-Sepharose high performance column as described above. Peak fractions were dialyzed against PBS and concentrated to 1 mg/ml. Purity and identity of the MCP-1 mutants were confirmed by silver-stained SDS-PAGE and nano-HPLC ESI-MS/MS, respectively.
Fluorescence Spectroscopy—Steady-state fluorescence measurements were performed on a Jasco FP6500 spectrofluorimeter (Japan) LS50B fluorometer and analyzed using the program Origin (Microcal Inc., Northampton, Mass.). The temperature was maintained at 20° C. during all experiments by coupling to an external water bath.
Isothermal Fluorescence Titration Experiments (IFT)—The emission spectra of 1 μM solution of each mutant in PBS were recorded over the range of 300-400 nm upon excitation at 282 nm. The excitation and emission slit widths were set at 3 and 5 nm, respectively. The spectra were recorded at a speed of 500 nm/min. The addition of GAG aliquots was followed by an equilibration period of 1 min before the next spectrum was recorded. After background subtraction, the spectra were integrated and the normalized mean changes in fluorescence intensity (−ΔF/F₀) obtained from three independent experiments were averaged and plotted against the volume corrected concentration of the added ligand. The resulting binding isotherms were analyzed by non-linear regression to an equation describing a bimolecular association reaction as described elsewhere (44).

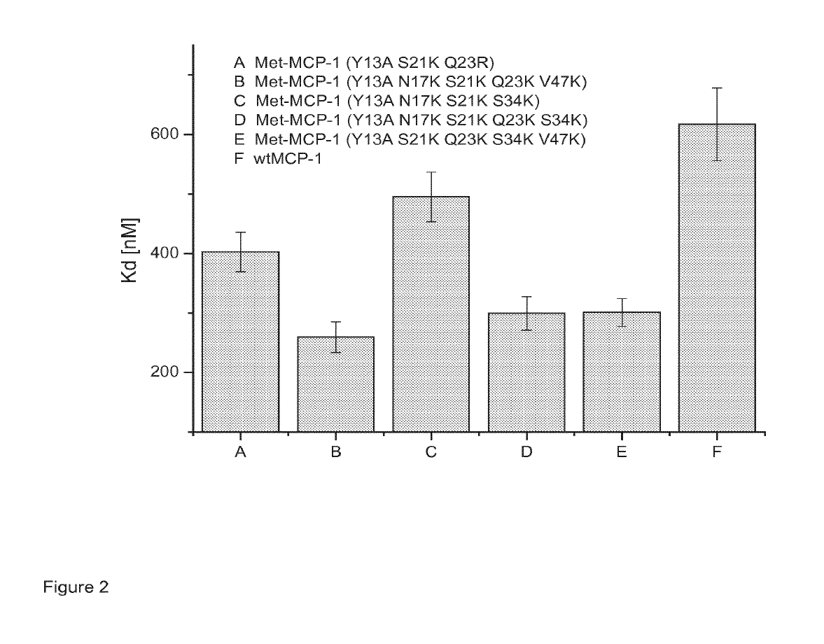

The results are shown in FIG. 2.

### Example 2

Surface Plasmon Resonance Analysis-Binding of wtMCP-1/CCL2 and MCP-1 mutants of the invention to unfractionated heparan sulfate was investigated on a BIAcore X100 instrument (BIAcore AB, Uppsala, Sweden). The immobilisation of the biotinylated heparin onto a streptavidin coated SA sensor chip was performed according to an established protocol, described recently. The actual binding interactions were recorded at 25° C. in PBST pH 7.4 containing 0.05% (v/v) Tween20 surfactant (BIAcore AB). 15 min injections of different protein concentrations at a flow rate of 30 μl/min with contact times between 60s and 120s which were followed by dissociation periods of 60s-120s in buffer and a pulse of 1M sodium chloride for complete regeneration. The maximum response signals of protein binding to the GAG surface, corresponding to the plateaus of the respective sensorgrams, were used for Scatchard plot analysis and the calculation of equilibrium dissociation constants (Kd values). The experiments were all carried out at a protein concentration ranging from 1 nM to 1.5 μM. Using the BlAevaluation software, koffs were calculated by separately fitting the dissociation phases according to the 1:1 steady-state interaction model.

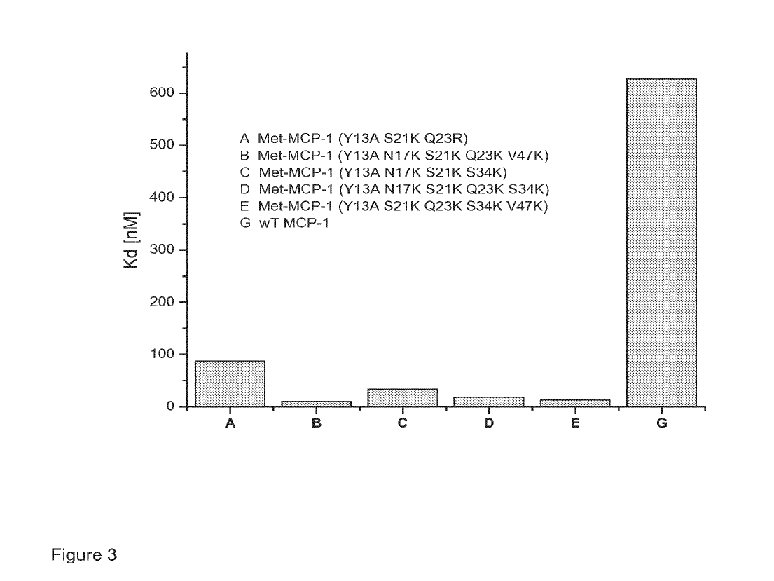

The results are shown in FIG. 3.

### Example 3

### Inhibitory Effects of MCP-1 Mutants on Inflammatory Monocyte Infiltration in the Thioglycolate Induced Peritonitis Model

The thioglycolate (TG)-induced peritonitis model in mice is an acute inflammatory model, characterized by monocyte/macrophage infiltrates, peaking about 16-24 hours after TG injection.

Therefore, to assess the anti monocyte migration activity in vivo this model can be selected.

One mL of sterile thioglycolate broth (4%) is injected i.p. in the absence or presence of 1 μg or 50 μg/mouse of MCP-1 mutant protein and lavages are harvested after 16 hours and analyzed by flow cytometry.

### Example 4

### Improvement of Experimental Autoimmune Uveitis by MCP-1 Mutants

Uveitis is an inflammatory autoimmune disorder of the inner eye and one of the major causes of blindness in industrialized countries. Animal models of Uveitis share many features with the human disease and therefore have helped in understanding the pathophysiology of uveitis and allowed the evaluation and introduction of new therapeutic treatment and regimens.

Experimental autoimmune Uveitis (EAU) in Lewis rats is mediated by CD4+T cells with specificity for retinal antigens. Once they have entered the eye they secrete cytokines and chemokines, which attract leukocytes to the eye. These inflammatory infiltrates, mainly monocytes/macrophages, are responsible for the damage of intraocular tissues.

The effect of MCP-I mutants is, therefore, tested in an experimental rat model of autoimmune Uveitis. Active immunisation is achieved by applying the uveitogenic peptide PDSAg, derived from the bovine retinal S-Antigen, into both hind legs in Freund's complete adjuvant (FCA), fortified with Mycobacterium tuberculosis strain H37RA. This leads to an initial activation of T cells which subsequently migrate and infiltrate into the eye. This phenomena starts to occur around day 8 post immunization. Groups of 5 Lewis rats are treated with MCP-1 mutants at a dose of 100 μg/animal dissolved in PBS or by PBS only using daily IP injections from day 1 after active immunization until day 22. The time course of disease is determined by daily examination of animals with an ophthalmoscope and Uveitis graded clinically as average clinical score of both eyes/group animals/day.

### Example 5

### Improvement of Parameters of Restenosis in apoE^{-/-} Mice by MCP-1 Mutants

Atherosclerosis with its clinical manifestation of myocardial infarction is close to becoming the leading cause of death worldwide. Atherosclerosis is a chronic inflammatory disease of the arterial wall characterized by an influx of mononuclear cells, which release cytokines and chemokines enhancing its recruitment and activation. Therefore, impairing monocyte recruitment should represent a valuable therapeutic target for the future clinical approach.

We test the ability of MCP-1 mutants to impact allmarks of atherosclerosis in the ApoE^{-/-} mice.

ApoE^{-/-} mice on atherogenic diet are subjected to a wire-injury model of the common carotid artery, and endothelial denudation will be achieved by 3 passes along the vessel. The mice are treated i.p. with 10 μg MCP-1 mutant dissolved in PBS or vehicle (PBS), one day before injury and then every day for 3 weeks. After 3 weeks, carotid arteries are excised after in situ perfusion-fixation with 4% paraformaldehyde and embedded in paraffin.

At the same time, carotid arteries are isolated on different animals 24 hours after denudation for ex vivo perfusion with MOPS-buffered physiological salt solution using a syringe pump. Monocytic MonoMac6 cells (500.000/mL) are labeled with Calcein-AM (Molecular Probes) and perfused at 5 μL/min after preincubation of the carotid artery with 1 μg/ml, 5 μg/ml and 10 μg/ml MCP-1 mutant protein. MonoMac6 adhesive interactions with the injured vessel wall (arrest, rolling) are recorded using stroboscopic epifluorescence illumination

### Example 6

### Improvement of Parameters of Myocardial Infarction by MCP-1 Mutants

Further investigation of the activity of MCP-1 mutant protein in a mouse model of myocardial infarction (MI) is performed. Indeed, myocardial infarction triggers also a complex inflammatory reaction characterized by cytokines and chemokines increase, which lead to monocyte recruitment at the ischemic site. Studies made in animal models with impaired monocyte infiltration show a reduction of the neointima formation and a preserved heart function after experimental induction of the myocardial infarction.

MI is induced in control and MCP-1 mutant protein treated group of mice by 30 min of proximal ligation of the left anterior descending artery. Mutant MCP-1 at the doses of 10, 5 and 1 μg is given IP 10 min and 2 hrs after surgical procedure, and will be followed by daily treatment for seven days (group size: n=5).

### Example 7

### MCP-1 Mutant Efficacy in Animal Models of Multiple Sclerosis

Multiple sclerosis (MS) is the commonest inflammatory demyelinating disease of the human central nervous system (CNS). MS pathology is characterized by breakdown of the blood-brain barrier (BBB), accompanied by infiltration of macrophages and T lymphocytes into CNS. The migration of these cells into the CNS parenchyma appears to be at least partly regulated by chemokines, among which MCP-1 seems to play a major role.

Expression and cellular localization of MCP-1 and CCR2 in MS have been described in the three compartments: brain, cerebrospinal fluid and blood.

Particularly important is the reported observation that in active demyelinating as well as in chronic active MS lesions, reactive hypertrophic astrocytes are strongly immunoreactive for MCP-1, suggesting a significant role for MCP-1 in the recruitment and activation of myelin-degrading macrophages and thereby contributing to the evolution of MS. In the same study, perivascular and parenchymal foamy macrophages do not express MCP-1 protein. Since foamy macrophages demonstrate a phenotype resembling that of anti-inflammatory M2 macrophages, are likely to contribute to resolution of inflammation and may therefore be responsible for inhibiting further lesion development and promoting lesion repair. Therapeutic approaches targeting MCP-1 could therefore be specific for inflammatory M1, not affecting anti-inflammatory/repairing M2 macrophages.

MCP-1 mutant protein can be tested in the rat-MOG₃₅₋₅₅-induced chronic EAE in C57BL/6 female mice. The treatment with 40, 200 and 400 μg/kg by intraperitoneal route (about 1, 5 and 10 μg/mouse) is started on day 7 post-immunization, when no clinical sign of the pathology are present, but an increase in circulating chemokine and chemokines (including JE: murine MCP-1) is already reported, and is continued for 21 days. Dexamethasone 1 mg/kg administered with the same administration route and regimen as mutant MCP-1 can be used as reference compound.

Body weight and clinical score are monitored daily in order to assess animal well being and degree of disease progression. Spinal cord and brain are collected to allow histological analysis for assessment of treatment effect on inflammatory infiltrates and demyelination based on positive effects on clinical score (TBA). A follow up experiment will aim to assess MCP-1 mutant protein activity on preventing/reducing relapses rate and severity in a model of relapsing remitting EAE. For this purpose MCP-1 mutant protein will be administered daily in the PLP-SJL mouse relapsing-remitting EAE model. Daily readouts and endpoint will be as in the previous experiment, but number and duration of relapses and remissions will also be calculated.

1. A MCP1 mutant protein with increased GAG binding affinity and reduced GPCR activity compared to wild type MCP-1 protein, characterized in that the MCP-1 protein is modified in a structure-conserving way by replacement of at least two amino acids by basic and/or electron donating amino acids, wherein at least one amino acid at positions 17 or 34 and optionally at least one amino acid at positions 21, 23 or 47 according to the numbering of SEQ ID NO: 1 is modified.

2. The MCP-1 mutant protein according to claim 1, characterized in that amino acids at positions 17 and 34 are modified.

3. The MCP-1 mutant according to claim 1, characterized in that the amino acid at position 21 and at least one of the amino acids at positions 17 or 34 are modified.

4. The MCP-1 mutant protein according to claim 1, characterized in that at least one amino acid of the first 1 to 10 amino acids of the N-terminal region of the wild type MCP-1 protein is modified by addition, deletion and/or replacement of at least one amino acid.

5. The MCP-1 mutant protein according to claim 1, characterized in that the modification in a structure conserving way is a deviation of the structure of the modified protein from wild type MCP1 structure of less than 30% as measured by far-UV CD spectroscopy.

6. The MCP-1 mutant protein according to claim 1, characterized in that the replacement of at least two amino acids comprises replacement with a basic amino acid selected from the group consisting of R, K, and H.

7. The MCP-1 mutant protein according to claim 1, characterized in that the replacement of at least two amino acids comprises replacement with an electron donating amino acid selected from the group consisting of N and Q.

8. The MCP-1 mutant protein according to claim 1, characterized in that Y at position 13 is substituted by A.

9. The MCP-1 mutant protein of claim 1, containing an N-terminal Met.

10. The MCP-1 mutant protein of claim 1, wherein the N-terminal amino acid residues 2-8 are deleted.

11. A MCP-1 mutant protein, characterized in that it comprises an amino acid sequence of the following formula:
(M)ₙQ(PDAINA(Z1))ₘVTCC(X1)NFT(X2)(Z2)
(Z3)I(X3)V(X4)RLASYRRITS(X5)
KCPKEAVIFKTI(X6) AKEICADPKQ KWVQDSMDHL DKQTQTPKT,
wherein Z1 is selected from the group consisting of P, A, G, and L,
wherein Z2 is selected from the group consisting of R and K,
wherein Z3 is selected from the group consisting of K and R,
wherein X1 is selected from the group consisting of Y and A,
wherein X2 is selected from the group consisting of N, R, K, H, N and Q,
wherein X3 is selected from the group consisting of S, K, H, N and Q,
wherein X4 is selected from the group consisting of R, K, H, N and Q,
wherein X5 is selected from the group consisting of S, K, H, N and Q,
wherein X6 is selected from the group consisting of V, R, K, H, N and Q,
wherein n and/or m can be either 0 or 1, and
wherein at least two of amino acids X1 to X6 are modified with the proviso that at least one of positions X2 and X5 is modified.

12. MCP-1 mutant protein selected from the group consisting of Met-MCP-1 Y13A N17K S21K Q23K V47K, Met-MCP-1 Y13A N17K S21K S34K, Met-MCP-1 Y13A N17K S21K Q23K S34K and Met-MCP-1 Y13A S21K Q23K S34K V47K.

13. An isolated polynucleic acid molecule, characterized in that the polynucleic acid molecule codes for a protein according to claim 1.

14. A vector, characterized in that the vector comprises an isolated DNA molecule according to claim 13.

15. A recombinant non-human cell, characterized in that the cell is transfected with a vector according to claim 14.

16. A pharmaceutical composition, characterized in that it comprises a protein according to claim 1 and a pharmaceutically acceptable carrier.

17. (canceled)

18. A method for treating a chronic or acute inflammatory disease or an autoimmune condition, comprising the step of administering the MCP-1 mutant protein according to claim 1 to a patient in need thereof.

19. The method according to claim 18, characterized in that the inflammatory disease is selected from the group consisting of rheumatoid arthritis, uveitis, inflammatory bowel disease, myocardial infarction, congestive heart failure, ischemia reperfusion injury, multiple sclerosis and atherosclerosis.

20. The MCP-1 mutant protein according to claim 5, characterized in that the modification in a structure conserving way is a deviation of the structure of the modified protein from wild type MCP1 structure of less than 20% as measured by far-UV CD spectroscopy.

21. The MCP-1 mutant protein of claim 11, wherein Z1 is A, X1 is A, X2 is K, X3 is K, X4 is K or R, X5 is K, and X6 is K.

22. The MCP-1 mutant protein of claim 11, wherein at least one of positions X1, X3 and X4 is modified.

23. A pharmaceutical composition, characterized in that it comprises a polynucleic acid molecule according to claim 13 and a pharmaceutically acceptable carrier.

24. A pharmaceutical composition, characterized in that it comprises a vector according to claim 14 and a pharmaceutically acceptable carrier.

25. A method for treating a chronic or acute inflammatory disease or an autoimmune condition, comprising the step of administering the polynucleic acid molecule according to claim 13 to a patient in need thereof.

26. A method for treating a chronic or acute inflammatory disease or an autoimmune condition, comprising the step of administering the vector according to claim 14 to a patient in need thereof.
